(19) United States
(12) Patent Application Publication
(10) Pub. No.: US 2004/0111103 A1
(43) Pub. Date: Jun. 10, 2004

(54) Surgical tape tensioner

(76) Inventor: Margaret Marie Nixon, Sunnyvale, CA (US)

Correspondence Address: CARSTENS YEE & CAHOON, LLP, P O BOX 802334, DALLAS, TX, 75380

(21) Appl. No.: 10/726,767

(22) Filed: Dec. 3, 2003

Related U.S. Application Data
(60) Provisional application No. 60/430,602, filed on Dec. 3, 2002.

(51) Int. Cl. A61B 17/08

(52) U.S. Cl. 606/151

(57) ABSTRACT

A tensioner for securing and tensioning surgical tape is made of a unitary piece of plastic having an opening by which the tensioner can be attached to a section of tape. The tensioner is attached to the surgical tape prior to use; once a vessel is to be occluded, a free end of the tape is wrapped around the vessel and tensioned until bleeding stops, then the surgical tape is secured in a gripping portion of the tensioner.

### CROSS-REFERENCE TO RELATED APPLICATION

This application claims the benefit of and priority to a U.S. Provisional Patent Application No. 60/430,602 filed Dec. 3, 2002, the technical disclosure of which is hereby incorporated herein by reference.

### BACKGROUND OF THE INVENTION

1. Technical Field

The present invention relates to an improved device to tension or secure surgical tape that is used in the surgical ligation of circulatory vessels or in positioning organs and structures during surgery.

3. Description of Related Art

During surgical procedures, there is a need to maintain a clear field of view of the affected anatomical structures for the surgeon. This can involve retracting intervening structures to one side of the operative field and stopping the leakage of blood or other fluids from their respective vessels. For tiny blood vessels that can regenerate quickly, the surgeon may choose to cauterize (seal with heat) a vessel, but with larger circulatory vessels, the surgeon must preserve the ability to restore normal circulation after surgery is completed, and will bind or ligate the vessel with surgical tape. Due to the possibility of causing injury to the circulatory vessel during the procedure, the surgical tape is preferably Retract-O-Tape® vascular loops, an air-cushioned tape that is designed to avoid injury to the vessel, and manufactured by the assignee of the current application. Examples of this tape are seen in FIGS. 1A and 2A.

Retract-O-Tape was first disclosed in U.S. Pat. No. 4,140,125, commonly owned by the assignee, which is hereby incorporated by reference. As seen in FIG. 1A, the tape 100 is formed as a round, hollow, silicone tube 110 that is then sealed at both ends with a silicone plug 116. The ambient air that becomes entrapped within the hollow core provides a cushioning effect on the anatomical structure and maintains the rounded cross-section of the tube during use. FIG. 1B shows a cross-section of the ends of the surgical tape, demonstrating the walls of the silicone tube 110, the hollow core 118, and the silicone plugs 116. The surgical tape is provided in different gauges for use with different size structures. FIG. 2A shows the same surgical tape 200 supplied with a blunt or pointed needle 202 at one end, which allows the surgeon to ligate a vessel without dissecting the vessel away from surrounding tissues. The end opposite the needle contains a silicone plug 216 to maintain the air cushioning. FIG. 2B shows a cross-section of the attachment of the needle 202 to the surgical tape 200.

FIGS. 3A-C show the process of a blood vessel being ligated. In FIG. 3A, a hemostat 320 holds a blunt needle 302 attached to a length of surgical tape 300 as the surgeon passes the needle 302 under a blood vessel 306. The tape 300 is then passed again in the same direction under the blood vessel, as seen in FIG. 3B, so that the end product, shown in FIG. 3C is a vessel 306 twice wrapped with the surgical tape 300. Although the patent that disclosed the air-cushioned tape also disclosed a tensioner to be used in conjunction with the tape, the tensioner was never brought to market, possibly because the tiny pieces of the tensioner could become separated and easily lost in the surgical field. Instead, a hemostat can be used to secure the free ends of the surgical tape. If the need for retraction or ligation is for a short time only, a surgical nurse may hold the hemostat, but for longer periods, the hemostat will be fastened to the surgical drapings or another object to maintain the necessary tension.

FIGS. 4A-D show details of the tensioner 400 disclosed in U.S. Pat. No. 4,140,125. The tensioner is composed of two separate pieces: the first piece, a clamping jaw 410 is seen in FIG. 4A in an open position. The clamping jaw 410 is made of a stiff but resilient plastic material, and is shaped to have two arms 416, 418, joined by a bendable hinge section 417. An integrally formed ridge 412 extends along a portion of the inner edge of one jaw 416 and fits into a mating groove 414 on the inner side of the opposite jaw 418 to prevent the jaws 416, 418 from laterally slipping in relation to one another when closed. FIG. 4B shows the clamping jaw 410 in a closed position, along with sleeve 420, which is made of an elastomeric material. In this closed position, sleeve 420 fits over the clamping jaw 410 to hold the clamping jaw closed. Note that a circular opening 422 is formed in the area of the hinge 417.

FIG. 4C shows the tensioner or clamping pad 400, formed of pieces 410 and 420, assembled with a length of surgical tape 402 running through the opening 422. Note that after assembly, a portion of each jaw 416, 418 protrudes from the enclosing sleeve 420. The tensioner 400 is shown in its anticipated use in FIG. 4D. Here, a blood vessel 450, seen in cross-section, is held between the surgical tape 402 and the sleeve 420 of tensioner 400. One end of the tape 402 is held in the opening 422 at the hinge end of the clamping jaws, while the other end has been fed between the protruding portion of the jaws 416, 418, where it would be firmly held.

It would be desirable to have a small tensioner, such as was originally intended, to use with the surgical tape instead of using hemostats. However, it would be desirable that such a tensioner not contain further small parts that could separate and cause problems. It is further desirable that such a tensioner be easily attachable to the surgical tape, easily used, and not easily lost.

### SUMMARY OF THE INVENTION

The present invention discloses a tensioner made of a unitary piece of plastic and designed to be used with a length of surgical tape. The tensioner is preferably supplied attached to a length of surgical tape that has a needle; design features allow the tape to be tensioned around a blood vessel or other elongated structure. In use, a surgeon uses the attached needle to pass the surgical tape around a blood vessel, such as the coronary artery, then threads the needle through a hole in the tensioner, so that the blood vessel is held between the side of the tensioner and the surgical tape. To stop bleeding, the surgical tape is tightened to provide sufficient tension between the tensioner and the tape, then the tape is secured in the slot opening off the hole in the tensioner. In this manner, the blood vessel is ligated, yet the ligation device takes very little room in the surgical field.

### BRIEF DESCRIPTION OF THE DRAWINGS

The novel features believed characteristic of the invention are set forth in the appended claims. The invention itself, however, as well as a preferred mode of use, further objectives and advantages thereof, will best be understood by reference to the following detailed description of an illustrative embodiment when read in conjunction with the accompanying drawings, wherein:

FIGS. 1A and 1B show an exemplary piece of surgical tape that the inventive device is designed to work with and a cross-section of the ends of that tape.

FIGS. 2A and 2B show another exemplary piece of surgical tape and a cross-section, this tape having a needle.

FIGS. 3A, 3B and, 3C demonstrate surgical tape being applied to ligate a blood vessel.

FIGS. 4A, 4B, 4C, and 4D show the parts and the use of a clamping pad according to the prior art.

FIG. 5 is a perspective view of a tensioner according to an embodiment of the present invention.

FIG. 6 shows a top view of the tensioner of FIG. 5, while FIGS. 6A through 6D show cross-sections and a detail of the embodiment of the innovative tensioner.

FIG. 7 shows a tensioner according to the present invention as it is used to ligate a blood vessel.

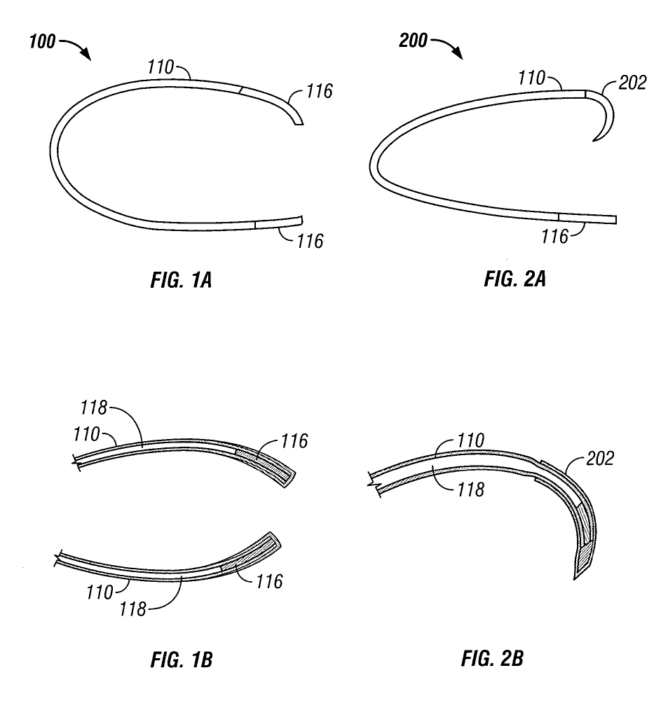

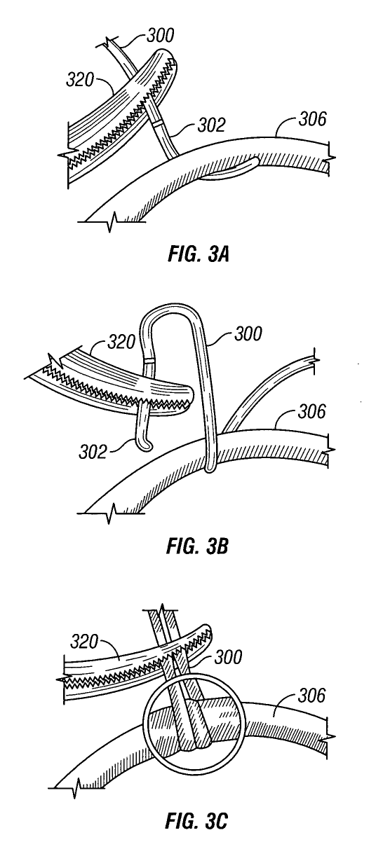

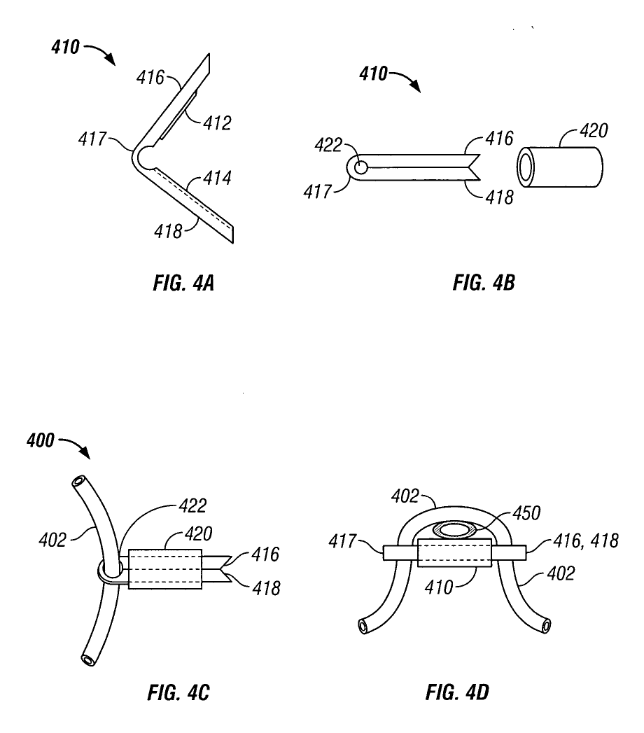

### DETAILED DESCRIPTION OF THE INVENTION

The description of the present invention has been presented for purposes of illustration and description, and is not intended to be exhaustive or limited to the invention in the form disclosed. Many modifications and variations will be apparent to those of ordinary skill in the art. The embodiment was chosen and described in order to best explain the principles of the invention, the practical application, and to enable others of ordinary skill in the art to understand the invention for various embodiments with various modifications as are suited to the particular use contemplated.

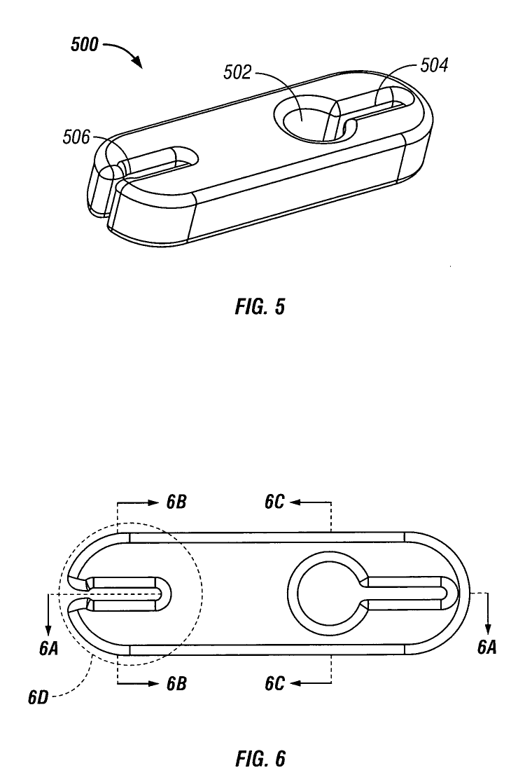

The present innovations are described with reference to FIGS. 5-7. FIG. 5 shows a greatly enlarged perspective view of an innovative tensioner 500 according to the disclosed invention. The tensioner 500 has generally rounded ends and edges are beveled to reduce the chance of injury to surrounding tissue when in use. A circular opening 502 extends through the tensioner 500. This opening is sized to permit a length of surgical tape and attached needle to pass through. Slot 504 opens off of the circular opening 502, while at the opposite end of the tensioner 500, slot 506 is similarly sized. Both of these slots 504, 506 provide a tight fit through which the surgical tape will not easily move.

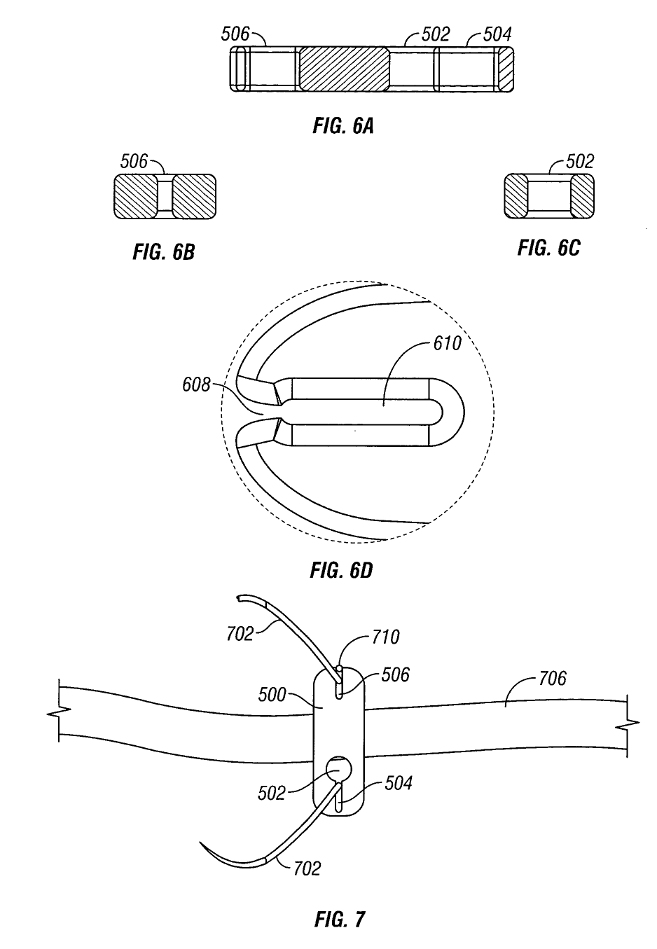

FIG. 6 is a top down view of the tensioner of FIG. 5 and shows the relative dimensions of an exemplary embodiment. The embodiment of the tensioner has an overall length of 0.310 inches and a width of 0.10 inches, with straight sides and essentially semi-circular ends. The circular opening has a diameter that is approximately one-half the width of the tensioner or 0.05 inches. FIG. 6A is a cross-section, along the length of the tensioner at its midline, with shaded areas showing where cuts would be made through the material of the tensioner to achieve the section. FIG. 6B shows a cross-section across the tensioner through slot 506, showing the narrow opening; FIG. 6C is a cross-section across the tensioner through the center of opening 502. FIG. 6D is an enlarged view of the slot 506. One can see most clearly in this view that the opening into slot 506 tapers inward to a neck 608, then opens out somewhat into the body 610 of the slot. This allows the surgical tape to be smoothly guided into the body 610, while preventing the tape from moving back out through the neck 608.

After manufacture of the tensioner, a short length of surgical tape, having an attached needle, is introduced into slot 506. Preferably the opening into slot 506 is then occluded, such as with a bead of adhesive. In this manner, the tensioner 500 is permanently attached to the surgical tape.

FIG. 7 shows the inventive tensioner in use. In this drawing, blood vessel 706 has been ligated between tensioner 500 and surgical tape 702. The surgical tape 702 is securely held in the two slots 504, 506. Adhesive bead 710 retains the tape in slot 506.

While the disclosed design is the presently preferred embodiment of the invention, one of ordinary skill in the art will understand that many variations on this specific design are possible without departing from the spirit of the invention.

1. A tensioner for a surgical tape, said tensioner comprising:
a unitary tensioner body, said tensioner body having
attaching means whereby said tensioner can be attached to a point on a length of surgical tape; and
holding means whereby said tensioner restrainably engages a second point of said surgical tape, the first and second points being longitudinally separated from each other along the length of surgical tape.

2. The tensioner of claim 1, wherein said tensioner is formed of plastic.

3. The tensioner of claim 1, wherein said attaching means comprises an elongated slot through the said unitary tensioner body.

4. The tensioner of claim 1, wherein said holding means comprises a circular opening through said unitary tensioner body.

5. The tensioner of claim 1, wherein said holding means comprises an elongated slot through the said unitary tensioner body.

6. A tensioner for a surgical tape comprising a unitary tensioner body having a first opening and a second opening passing therethrough,
wherein said first opening has a first section having size and shape such that a surgical tape of a given size can frictionally move through said first opening and a second section having a size and shape to restrainably engage the surgical tape of a given size,
wherein said second opening has a size and shape to restrainably engage the surgical tape of a given size.

7. The tensioner of claim 6, wherein said tensioner is formed of plastic.

8. The tensioner of claim 6, wherein said first section of said first opening has a circular cross-section.

9. The tensioner of claim 6, wherein said second section of said first opening is an elongated slot.

10. The tensioner of claim 6, wherein said second opening is an elongated slot.

11. A method of clamping an elongated anatomical structure, such as a blood vessel, the method comprising the steps of:
attaching a tensioner, having a unitary body, to a length of surgical tape;
passing a portion of said surgical tape around the elongated body structure; and
securing said surgical tape in place by inserting a section of said surgical tape into a gripping portion of said tensioner.

12. The method of claim 11, wherein said attaching step comprises securing a free end of said length of surgical tape in a slot in said tensioner.

13. The method of claim 11, wherein said securing step comprises threading said length of surgical tape through an opening in said tensioner.
